(19) United States
(12) Patent Application Publication
(10) Pub. No.: US 2003/0109745 A1
(43) Pub. Date: Jun. 12, 2003

(54) Method for producing carboxylic acid benzyl esters

(76) Inventors: Pieter Ooms, Krefeld (DE); Bernd-Ulrich Schenke, Bottrop (DE)

Correspondence Address: BAYER CHEMICALS CORPORATION, 100 BAYER ROAD, PITTSBURGH, PA, 15205, US

(21) Appl. No.: 10/276,872

(22) PCT Filed: May 7, 2001
(86) PCT No.: PCT/EP01/05138

(30) Foreign Application Priority Data
May 19, 2000 (DE) 100 24 936.1

(51) Int. Cl. C07C 67/24

(52) U.S. Cl. 560/240

(57) ABSTRACT

The invention relates to a method for producing carboxylic acid benzyl esters from dibenzyl ethers.

The invention relates to a method for producing carboxylic acid benzyl esters from dibenzyl ethers.

Benzyl acetate, the main component of jasmin oil, is an important odor compound for producing fragrance compositions and is a starting material for the production of fruit ethers.

The production of benzyl acetate has already been reported several times. Thus, for example, producing benzyl acetate by reacting benzyl alcohol with acetic acid has long been known. Benzyl acetate can also be produced by reacting benzyl chloride with alkali metal acetates, in the presence or absence of phase transfer reagents (Wang et al., Chem. Eng. Commun. 100, (1991), 135-147). A disadvantage is the formation of salts which must be disposed of, and thus decrease the economic efficiency of this method.

DD-A5-286 577 describes the production of benzyl acetate by reacting dibenzyl ether with acetic anhydride. Disadvantages are the drastic reaction conditions (300° C./20 MPa) and the only moderate yields.

An object was therefore to provide a method starting from dibenzyl ether for producing carboxylic acid benzyl esters which can be carried out under mild reaction conditions and leads to good yields.

Surprisingly, a method has now been found for producing carboxylic acid benzyl esters from dibenzyl ethers, which is characterized in that dibenzyl ethers are reacted with carboxylic acids in the presence of a heterogeneous acid catalyst.

The dibenzyl ether used in the inventive method is an unsubstituted or substituted dibenzyl ether which can bear, for example, one or more substituents selected from the group of branched and unbranched C₁-C₆-alkyl, C₁-C₆-alkoxy, CN, CO(C₁-C₆)-alkyl, NO₂ and halogen. Preferred substituents are methyl, methoxy or chlorine. Particularly preferably, unsubstituted dibenzyl ether is used.

In the inventive method, dibenzyl ether or dibenzyl ether/benzyl alcohol mixtures, as arise, for example, in the production of benzyl alcohol from benzyl chloride can be used. The dibenzyl ether content in the dibenzyl ether/benzyl alcohol mixture can be, for example, 50 to 100% by weight, preferably 60 to 100% by weight, particularly preferably 70 to 100% by weight.

The carboxylic acids used in the inventive method are unbranched or branched alkyl, aryl or aralkyl carboxylic acids which are saturated or unsaturated and contain 1 to 50 carbon atoms, preferably 2 to 30 carbon atoms, particularly preferably 2 to 10 carbon atoms. In the inventive method, for example, formic acid, acetic acid, propionic acid, butyric acid, isobutyric acid, valeric acid, isovaleric acid, caproic acid, caprylic acid, lauric acid, myristic acid, stearic acid, oleic acid, acrylic acid, phenylacetic acid, benzoic acid or salicylic acid can be used. Very particularly preferred carboxylic acids are acetic acid and propionic acid.

The inventive method is preferably carried out with removal of the water formed. A suitable method for removing water is distillation or passing an inert gas, nitrogen for example, through the mixture. Preferably, to remove the water formed, dehydrating means are used, for example zeolites, aluminum oxides or clay earths. Particularly preferably the water formed is removed by means of the fact that the reaction is carried out in the presence of the corresponding anhydride of the carboxylic acid used, as dehydrating means. Very particularly preferred anhydrides are acetic anhydride and propionic anhydride.

In the inventive method, preferably 2 to 50 equivalents of carboxylic acid, more preferably 3 to 30 equivalents, particularly preferably 4 to 20 equivalents, based on dibenzyl ether, are used.

If the inventive method is carried out in the presence of the corresponding anhydride of the carboxylic acid used, preferably 0.1 to 10 equivalents of anhydride, preferably 0.5 to 7.5 equivalents, particularly preferably 1 to 5 equivalents, based on dibenzyl ether, are used. Since one molecule of anhydride used reacts to completion with uptake of water to form 2 molecules of carboxylic acid, in the inventive method smaller amounts of carboxylic acid can be used. Then, preferably 1 to 25 equivalents of carboxylic acid, preferably 1.5 to 15 equivalents, particularly preferably 2 to 10 equivalents, of carboxylic acid are used, based on dibenzyl ether.

In the inventive method, the heterogeneous acid catalysts used are preferably acid ion exchangers, for example polymers bearing sulfonic acid groups, where the polymers can be, for example, polystyrenes, styrene-divinylbenzene copolymers or phenol/formaldehyde resins. Preferred acid ion exchangers are sulfonylated polystyrenes, sulfonylated styrene-divinylbenzene copolymers or sulfonylated phenol-formaldehyde resins, very particularly preferably sulfonylated polystyrenes.

Furthermore, fluorinated or perfluorinated sulfonic-acid-group-bearing polymers are particularly preferably used, such as, for example, fluorinated or perfluorinated sulfonylated polystyrenes, fluorinated or perfluorinated sulfonylated styrene-divinylbenzene copolymers, or fluorinated or perfluorinated sulfonylated phenol-formaldehyde resins. Very particularly preferably, fluorinated or perfluorinated sulfonylated polystyrenes are used.

The sulfonic-acid-group-bearing ion exchangers can be prepared by reacting polymers with sulfonating agents such as sulfuric acid or chlorosulfonic acid. The preparation is described, for example, in Encyclopedia of Polymer Science and Technology Vol. 7, Ed. N. M. Bikales, Interscience Publishers New York, 1967, pp. 695 ff.

Mixtures of acidic ion exchangers can also be used.

Since Mastagli et al., C. r. 232, 1951, 1848-1849 disclose that dibenzyl ether is converted in the presence of sulfonated phenol-formaldehyde resins to form toluene and benzaldehyde, the suitability of acidic ion exchangers as catalysts in the inventive method is particularly surprising.

The acidic ion exchangers can be in bead form and have particle sizes of 0.3 to 3.0 mm in diameter. They can be of the gel type or macroporous. Their total capacity of acid functions in water-moist form having a water content of approximately 75 to 85% by weight is preferably 0.7 to 2.1 or 3.5 to 5 m-equivalents/ml of ion exchanger, based on 1 g of dry matter of ion exchanger.

Suitable acidic ion exchangers are, for example, the products distributed under the following registered tradenames Lewatit®, Amberlite®, Dowex®, Duolite®, Nafion®, Permutit®, Chempro® or Imac®.

In the inventive method, the acidic ion exchangers are preferably used in dried form. They can be dried by heat and/or vacuum. In addition, they can be dried by washing with hydrophilic liquids, such as the carboxylic acid used in the method, or the corresponding carboxylic anhydride, or by azeotropic distillation with organic solvents such as toluene, xylene or methylene chloride.

The heterogeneous acid catalyst, preferably an acidic ion exchanger, can be used in the inventive method in suspended form or as fixed-bed catalyst.

If the heterogeneous acid catalyst is used in suspended form, it is for preference used in an amount of 0.1 to 100% by weight, preferably 0.5 to 90% by weight, particularly preferably 0.1 to 80% by weight, based on dibenzyl ether. It is preferably employed with intensive mixing of the reaction partners. Intensive mixing can be achieved in various ways known to those skilled in the art, for instance via stirring, nozzles, baffles, static mixers, pumps, turbulent flow in narrow tubes or via ultrasound.

In a preferred embodiment of the inventive method, the heterogeneous acid catalyst, preferably an acidic ion exchanger, is suspended in the carboxylic acid used, preferably in a mixture of carboxylic acid used and the corresponding carboxylic anhydride, and then dibenzyl ether is added. After completion of the reaction, the suspended catalyst can be separated off, for example by filtration or centrifugation.

If the heterogeneous acid catalyst is used as a fixed-bed catalyst, for preference catalyst hourly space velocities of 0.05 to 5000 g of dibenzyl ether per liter of catalyst per hour are used, preferably 0.1 to 4000 g of dibenzyl ether per liter of catalyst per hour, particularly preferably 1.0 to 3000 g of dibenzyl ether per liter of catalyst per hour.

In a preferred embodiment of the inventive method, the heterogeneous acid catalyst, preferably an acidic ion exchanger, is a fixed-bed catalyst. This is preferably disposed as a catalyst bed in a tube. The starting materials dibenzyl ether and carboxylic acid, preferably in a mixture with the corresponding carboxylic anhydride, can be brought into contact with the catalyst by flooding in cocurrent or countercurrent flow.

In a further preferred embodiment of the inventive method, this is carried out in the trickle phase and the heterogeneous acid catalyst, preferably an acidic ion exchanger, is a fixed-bed catalyst. Preferably, the catalyst bed is situated in a vertically upright tubular reactor which preferably comprises intermediate plates for improved distribution of the liquid stream and for better wetting of the catalyst bed.

Preferably, the starting materials are applied cocurrently, for example from the top, onto a catalyst bed disposed in a tube. At the end of the tube the reaction products can be taken off.

The reaction products can be worked up, not only in the case of the suspended catalyst, but also in the case of the fixed-bed method variant, in such a manner that a water-immiscible solvent, preferably toluene, is added to the reaction products. After separating off the organic phase which contains the crude carboxylic acid benzyl ester, this can be further purified by distillation, for example.

The inventive method can be carried out batchwise, semicontinuously or continuously.

The temperature at which the inventive method is carried out is preferably 15 to 200° C., particularly preferably 25 to 190° C., very particularly preferably 30 to 180° C.

When the inventive method is carried out above about 115° C., in accordance with the vapor pressure, elevated pressure must be employed. The required superatmospheric pressure is then at least equal to the vapor pressure of the reaction mixture. It can be up to about 50 bar, preferably up to 25 bar.

If appropriate the inventive method can be carried out under a conventional protective gas, for example, nitrogen, helium or argon.

The inventive method produces carboxylic acid benzyl esters in good yields at a high conversion rate and with good selectivity. The inventive method can be carried out simply without great apparatus requirements.

The percentages in the examples which follow are based on weight.

### EXAMPLES

### Example 1

99.2 g (0.5 mol) of dibenzyl ether, 180.0 g (1.5 mol) of acetic acid and 3.0 g of Lewatit® SP 118 (Bayer AG) were heated to 100° C. in a flask equipped with baffle and blade stirrer with vigorous stirring (250 rpm) under nitrogen. After 7 h of reaction time the mixture was cooled rapidly, and the organic phase, after addition of toluene and water, was separated off and analyzed by gas chromatography.

The reaction mixture contained benzyl acetate and dibenzyl ether in a ratio of 14 to 82.

### Example 2

Example 2 was carried out in a similar manner to Example 1. 300.0 g (5.0 mol) of acetic acid and 3.0 g of Lewatit® SC 102 (Bayer AG) were used and the reaction was carried out at 120° C. The reaction time was 12 h.

The reaction mixture contained benzyl acetate and dibenzyl ether in a ratio of 82 to 4.

### Example 3

Example 3 was carried out in a similar manner to Example 1. 300.0 g (5.0 mol) of acetic acid and 3.0 g of Dowex® 50×4 (Dow Chemical) were used and the reaction was carried out at 120° C. The reaction time was 7 h.

The reaction mixture contained benzyl acetate and dibenzyl ether in a ratio of 50 to 44.

### Example 4

Example 4 was carried out in a similar manner to Example 1. 120.0 g (2.0 mol) of acetic acid and 3.0 g of Nafion® SAC 13 (Du Pont) were used and the reaction was carried out at 120° C. The reaction time was 7 h.

The reaction mixture contained benzyl acetate and dibenzyl ether in a ratio of 27 to 67.

### Example 5

Example 5 was carried out in a similar manner to Example 1. 99.2 g (0.5 mol) of dibenzyl ether, 30.0 g (0.5 mol) of acetic acid, 51.1 g (0.5 mol) of acetic anhydride and 3.0 g of Lewatit® SPC 118 (Bayer AG) were used. The reaction time was 20 min.

The reaction mixture contained benzyl acetate and dibenzyl ether in a ratio of 79 to 7.

### Example 6

Example 6 was carried out in a similar manner to Example 5. 3.0 g of Lewatit® SC 104 (Bayer AG) were used, the reaction time was 1 h.

The reaction mixture contained benzyl acetate and dibenzyl ether in a ratio of 77 to 4.

### Example 7

Example 7 was carried out in a similar manner to Example 5. 3.0 g of Amberlyst® (Acros) were used and the reaction was carried out at 120° C. The reaction time was 4 h.

The reaction mixture contained benzyl acetate and dibenzyl ether in a ratio of 62 to 24.

### Example 8

Example 8 was carried out in a similar manner to Example 5. 3.0 g of Dowex® 50×4 (Dow Chemical) were used, and the reaction time was 1 h.

The reaction mixture contained benzyl acetate and dibenzyl ether in a ratio of 71 to 10.

### Example 9

Example 9 was carried out in a similar manner to Example 5. 3.0 g of Nafion® NR 50 (Du Pont) were used, and the reaction time was 2 h.

The reaction mixture contained benzyl acetate and dibenzyl ether in a ratio of 80 to 4.

### Example 10

Example 10 was carried out in a similar manner to Example 5. 3.0 g of Nafion® SAC 13 (Du Pont) were used, and the reaction time was 30 min.

The reaction mixture contained benzyl acetate and dibenzyl ether in a ratio of 82 to 4.

### Example 11

Example 11 was carried out in a similar manner to Example 5. 37.0 g (0.5 mol) of propionic acid and 65.1 g (0.5 mol) of propionic anhydride were used, and the reaction time was 30 min.

The reaction mixture contained benzyl propionate and dibenzyl ether in a ratio of 76 to 8.

### Example 12 (continuous method)

In a reactor containing 85 parts by volume of sulfonated polystyrene resin Lewatit® SC 104 (Bayer AG) swollen in acetic acid (corresponding to about 45 parts by volume or 40 parts by weight dried at 60° C.) arranged as a fixed bed, at the top end of the reactor, a mixture of 22.6 parts by weight/h of dibenzyl ether and 27.4 parts by weight/h of acetic acid was passed through the catalyst bed at a temperature of 100° C. The reaction mixture leaving the reactor contained benzyl acetate and dibenzyl ether in a ratio of 52 to 39.

Space-time yield: 0.209 kg/lh (based on swollen ion exchanger) or 0.395 kg/lh (based on dry ion exchanger).

### Example 13 (continuous method)

Example 13 was carried out in a similar manner to Example 12. 45.6 parts by weight/h of dibenzyl ether and 54.6 parts by weight/h of acetic acid were used. The reaction mixture leaving the reactor contained benzyl acetate and dibenzyl ether in a ratio of 37 to 57.

Space-time yield: 0.301 kg/lh (based on swollen ion exchanger) or 0.569 kg/lh (based on dry ion exchanger).

### Example 14 (continuous method)

Example 14 was carried out in a similar manner to Example 12. 24.8 parts by weight/h of dibenzyl ether and 75.0 parts by weight/h of acetic acid were added to 66 parts by volume of sulfonated polystyrene resin Lewatit® SC 102 (Bayer AG) swollen in acetic acid (corresponding to about 32 parts by volume dried at 60° C.). The reaction mixture leaving the reactor contained benzyl acetate and dibenzyl ether in a ratio of 49 to 46.

Space-time yield: 0.276 kg/lh (based on swollen ion exchanger) or 0.569 kg/lh (based on dry ion exchanger).

### Example 15 (isolation)

Example 15 was carried out in a similar manner to Example 5. 3.0 g of Lewatit® SC 102 (Bayer AG) were used and the reaction was carried out at 60° C. The reaction time was 3 h.

After filtering off the Lewatit, the reaction mixture was separated by distillation. 93.4 g (62%) of benzyl acetate were isolated at 104 to 107° C./30 mbar. First runnings and last runnings contained a further 3.0 g (2%) of benzyl acetate.

1. A method for producing carboxylic acid benzyl esters from dibenzyl ethers, characterized in that dibenzyl ethers are reacted with carboxylic acids in the presence of a heterogeneous acid catalyst.

2. The method as claimed in claim 1, characterized in that the heterogeneous acid catalyst is an acidic ion exchanger.

3. The method as claimed in claim 1 or 2, characterized in that the heterogeneous acid catalyst is a sulfonic-acid-group-bearing polymer.

4. The method as claimed in one or more of the preceding claims 1 to 3, characterized in that the heterogeneous acid catalyst is a fluorinated or perfluorinated sulfonic-acid-group-bearing polymer.

5. The method as claimed in one or more of the preceding claims 1 to 4, characterized in that the dibenzyl ether is an unsubstituted dibenzyl ether.

6. The method as claimed in one or more of the preceding claims 1 to 5, characterized in that the dibenzyl ether is a substituted dibenzyl ether which bears one or more substituents selected from the group consisting of C₁-C₆alkyl, C₁-C₆-alkoxy, CN, CO(C₁-C₆-alkyl), NO₂ and halogen.

7. The method as claimed in one or more of the preceding claims 1 to 6, characterized in that dibenzyl ether is used in a mixture with benzyl alcohol.

8. The method as claimed in one or more of the preceding claims 1 to 7, characterized in that 2 to 50 equivalents of carboxylic acid, based on dibenzyl ether, are used.

9. The method as claimed in one or more of the preceding claims 1 to 8, characterized in that the reaction is carried out with removal of the water formed.

10. The method as claimed in claim 7, characterized in that the water formed is removed by distillation or by passing through an inert gas.

11. The method as claimed in one or more of the preceding claims 1 to 10, characterized in that the reaction is carried out in the presence of the corresponding anhydride of the carboxylic acid used.

12. The method as claimed in claim 11, characterized in that 0.1 to 10 equivalents of anhydride, based on dibenzyl ether, are used.

13. The method as claimed in one or more of the preceding claims 1 to 12, characterized in that the heterogeneous acid catalyst is suspended in the reaction mixture.

14. The method as claimed in claim 13, characterized in that the suspended catalyst is used in amounts of 0.1 to 100% by weight, based on dibenzyl ether.

15. The method as claimed in one or more of the preceding claims 1 to 12, characterized in that the heterogeneous acid catalyst is used as a fixed-bed catalyst.

16. The method as claimed in claim 15, characterized in that catalyst hourly space velocities of 0.05 g to 5000 g of dibenzyl ether per liter of heterogeneous acid catalyst per hour are used.

17. The method as claimed in one or more of the preceding claims 1 to 16, characterized in that the reaction is carried out at temperatures of 15 to 200° C.
